(19) United States
(12) Plant Patent Application Publication
(10) Pub. No.: US 2016/0205831 P1
(43) Pub. Date: Jul. 14, 2016

(54) White delight four

(50) ;

(71) Applicant: The Texas A&M University System, College Station, TX (US)

(72) Inventors: David H. Byrne, Bryan, TX (US); Natalie Anderson, Calvert, TX (US)

(73) Assignee: The Texas A&M University System, College Station, TX (US)

(21) Appl. No.: 14/544,505

(22) Filed: Jan. 13, 2015

(51) Int. Cl. A01H 5/00

(52) U.S. Cl. PLT/195

(57) ABSTRACT

Disclosed is a new variety of Prunus persica named ‘White Delight Four’. This new variety, which requires approximately 700 chilling units of dormancy, is considered to be a peach tree of mid to late mid season maturity, which produces white fleshed, sub-acid fruit that are very firm, attractively colored, and suitable for both local and regional markets.

### BACKGROUND OF THE INVENTION

### FIELD OF THE INVENTION

This invention relates to peach trees and, more specifically, to peach trees referred to as a variety of Prunus persica named ‘White Delight Four’ ‘White Delight Four’, which requires approximately 700 chilling units of dormancy, produces an high quality, subacid, large, firm, clingstone, white-fleshed peach that matures in early July in the medium chill zone of Texas.

### SUMMARY OF THE INVENTION

The ‘White Delight Four’ peach is characterized as to novelty and is otherwise noteworthy by producing fruit that ripens in early July in the medium chill zone of Texas; is considered very high quality; and which is firm and has an attractive coloration. In this regard, the present variety of peach tree bears fruit that are ripe for commercial harvesting and shipment in the first days of July, when the fruit is grown in the medium chill zone of Texas. ‘White Delight Four’ ripens about a month after ‘June Gold’ (Brooks, 1958) and 15-20 days after ‘Scarlet Pearl’ (Okie, 1993).

ORIGIN OF THE VARIETY

The present peach tree was the result of an ongoing Stone Fruit Breeding Program of Texas A & M University, College Station, Brazos County, Tex. To this end, both controlled and hybrid crosses are made each year in order to produce seedling populations from which improved progenies are evaluated and selected.

The seedling ‘White Delight Four’ was originated at the Texas A & M University Horticultural Farm in College Station, Tex. from a cross done in 1998 between a low chill, yellow fleshed peach selection, TXW1591-1, and a subacid, white nectarine developed in China, Zao Hong Zhu'. TXW1591-1 is a yellow-fleshed, acid peach derived from a cross between ‘Tropicsweet’ and the Florida selection FLA9-6N (Fla6-8 open pollinated). ‘Zao Hong Zhu’ was released in 1994 by the breeding program of the Beijing Academy of Agriculture and Forest Science, Plant Protection and Environment Institute and was created by crossing the subacid, white-fleshed ‘Jingyu’ (=‘Okubo’×‘Okitsu’) and the Arkansas selection A369 (=7761 open pollinated) (Lirong Wang, personal communication). A369 is a large late ripening nectarine selected in Arkansas in 1986 and derived from materials developed in the New Jersey Fruit Breeding Program directed by Drs. Fred Hough and Catherine Bailey (John Clark, personal communication; Joseph Goffreda, personal communication). Resulting seed from these crosses were planted in 1999 in a high density breeding orchard at the Texas A & M University Horticultural Farm in College Station, Tex. During 2001 a seedling was chosen from this hybrid population of seedlings as having exceptional characteristics. This seedling was denominated as TX4F194LW. Two-year and older trees of the variety were subsequently evaluated during the 2006 through 2011 fruit growing seasons in both California (Fowler) and Texas (College Station, Fairfield and Terrell).

### ASEXUAL REPRODUCTION OF THE VARIETY

‘White Delight Four’ was bud grafted onto virus-free Nemaguard (“The Brooks and Olmo Register of Fruit and Nut Varieties,” 3rd Ed., American Society of Horticultural Science Press, Alexandria, Va., 1997) peach rootstock at the nursery site in Oakdale, Calif. The variety was subsequently planted at the experimental orchard in the central portion of the San Joaquin Valley, near Fowler, Fresno County, Calif. and in three sites in Texas (College Station, Fairfield and Terrell). Fruit from the resulting propagation has been evaluated during the period from 2006 to 2011 fruit seasons. This evaluation clearly demonstrated that the re-propagated trees were true to the characteristics of the original seedling in all observable aspects.

### BRIEF DESCRIPTION OF THE DRAWINGS

This new variety of peach tree is illustrated by the accompanying photographic drawings and depicts the plant by the best possible color representation using color photography, wherein:

FIG. 1. Color photograph of the fruit with twigs and leaves of ‘White Delight Four’ in a tree in Fowler, Calif.

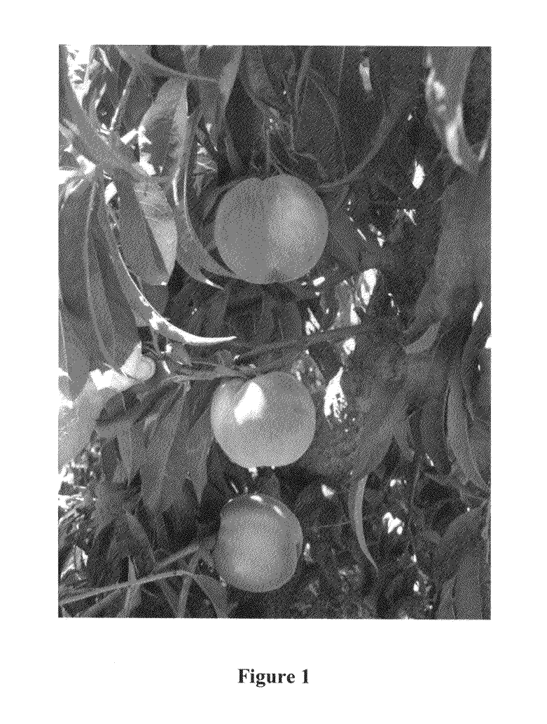

FIG. 2. Color picture showing the flesh and skin color and fruit shape of ‘White Delight Four’ produced in the medium chill zone of Texas (Fairfield).

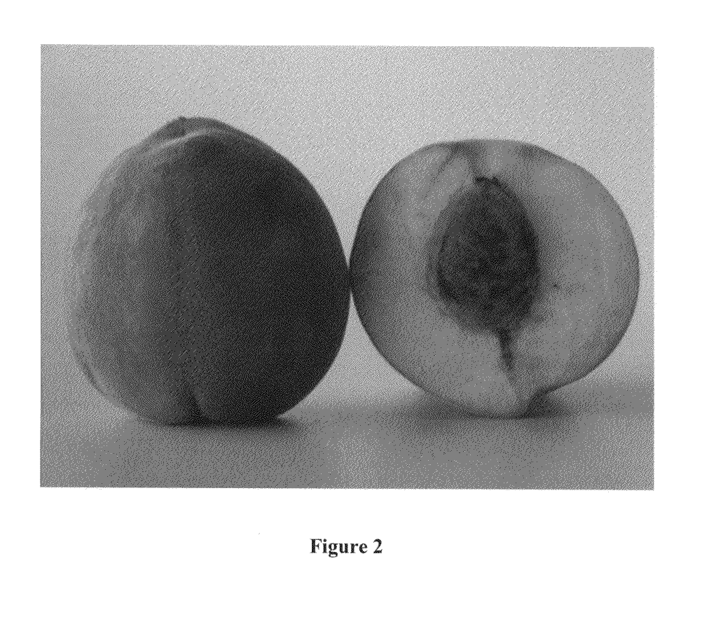

FIG. 3. Color photograph of the endocarp of ‘White Delight Four’. The ruler is demarcated in millimeters.

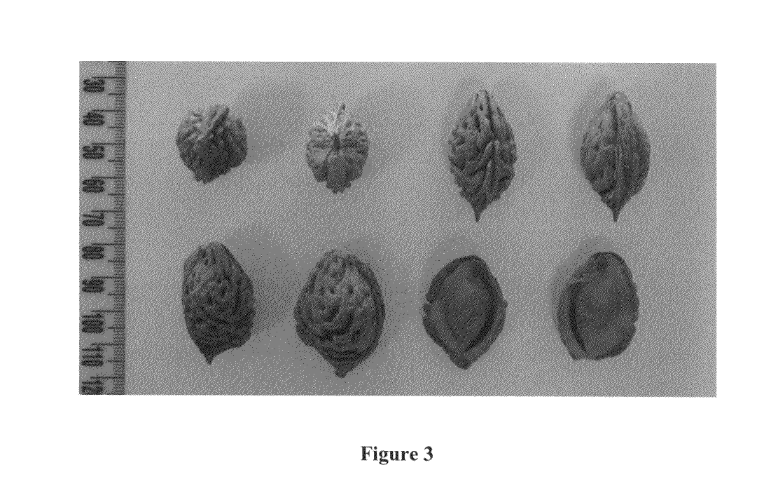

FIG. 4. A stem showing the leaves of the ‘White Delight Four’ peach. The ruler is demarcated in millimeters.

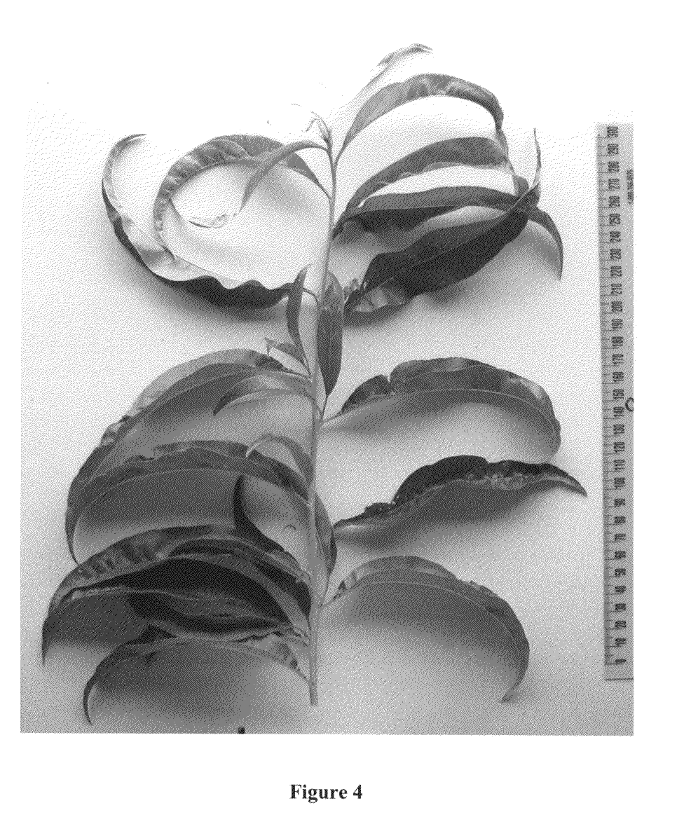

FIG. 5. Showy flowers of ‘White Delight Four’. The ruler is in millimeters.

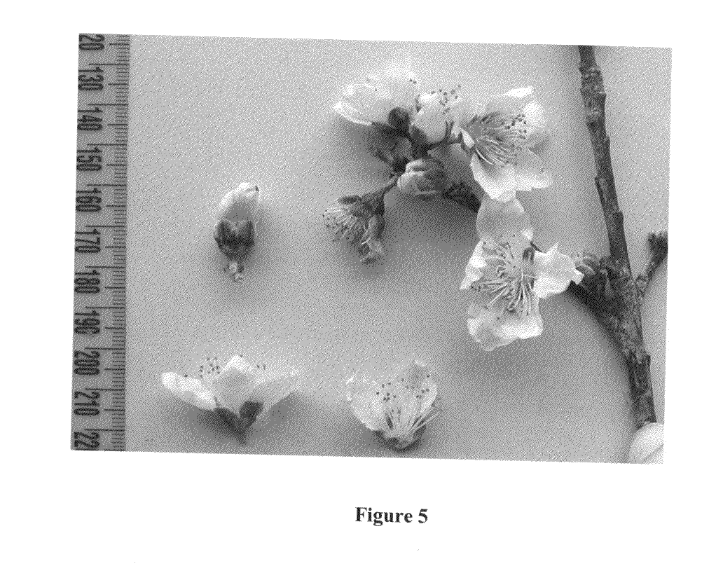

### BOTANICAL DESCRIPTION OF THE VARIETY

Referring more specifically to the pomological details of this new and distinct variety of peach tree, the following has been observed under the ecological conditions prevailing at the experimental orchards in the medium chill zone of Texas (Fairfield). All major color code designations are by reference to the RHS Colour Chart (Third Edition) provided by The Royal Horticultural Society of Great Britain. Colors are approximate as color depends on horticultural practices such as light level and fertilization rate, among others.
Tree:
   Size.—Generally average to above average as compared to other common peach cultivars ripening in the mid to late season of maturity.
   Height.—8 feet (2.44 m) at the end of the 2012 growing season.
   Width.—6 feet (1.83 m) at the end of the 2012 growing season.
   Vigor.—High.
   Density.—Medium to high.
   Productivity.—Productive.
   Shape.—The trees are vigorous with the typical semi-spreading growth habit similar to ‘TexKing’, ‘TexPrince’, and ‘TexRoyal’.
   Current season growth.—The current season growth for the new variety was approximately 3.8 to 4.1 feet (1.16-1.25 m).
   Regularity of bearing.—Regular, and considered hardy under typical climatic conditions found in the medium chill zone of Texas and in the central San Joaquin Valley, Calif.
Trunk:
   Size.—Approximately 3.3 inches (8.26 cm) in diameter and 11.8 inches (29.97 cm) in circumference when measured at a distance of approximately 12 inches (30.5 cm) above the soil level, at the end of the 2012 growing season on a five-year old tree.
   Bark texture.—Considered moderately rough with numerous folds of papery scarf-like skin being present.
   Bark coloration.—Variable, colors present are 165C and 166A of the Greyed-Orange Group, N200D of the Brown Group and 202D of the Black Group.
Branches:
   Size.—Considered medium for the variety.
   Thickness.—Average (about 5.8 cm in diameter as measured 10 cm from the trunk on a five-year old tree) as compared to other varieties.
   Surface texture.—Average and appearing furrowed on wood that is several years old.
   Lenticels.—Numerous flat, oval lenticels present. The lenticels range in size from approximately 3 to 8 mm in width and were approximately 1 mm in height.
   Current season shoots.—Surface texture — Substantially glabrous.
   Internode length.—Approximately 1.5 to 5.0 cm as measured in the middle of a current season stem.
   Color of mature branches.—The predominant colors are 166A Greyed-Orange Group, 187A of the Greyed-Purple Group, N200D of the Brown Group and 201D of the Grey Group.
   Current season shoots.—Color — Light green (143D and 144B-C of the Green Group) with some reddish-brown coloration appearing on exposed surface of the shoots (170A-B of the Greyed-Orange Group). The color of new shoot tips is considered a bright and shiny green (mainly Green Group 141C-D).
Leaves:
   Size.—Considered moderately large for the species. Leaf measurements have been taken from vigorous upright current season growth approximately at mid-shoot.
   Leaf length.—Approximately 147 to 175 mm.
   Leaf width.—Approximately 32 to 38 mm.
   Leaf thickness.—Less than 1 mm.
   Leaf form.—Lanceolate.
   Leaf tip form.—Acuminate.
   Leaf upper surface color.—Green, approximately 137B of the Green Group.
   Leaf lower surface color.—Green, approximately 137B and 138A of the Green Group.
   Leaf mid-vein color.—Varies from pale green to yellow green, approximately 138D of the Green Group and 145D Yellow-Green Group.
   Leaf margins.—
   Form.—Considered crenate/crenulate.
   Uniformity.—Considered generally uniform.
   Leaf petioles.—
   Size.—Considered medium long.
   Length.—Approximately 12 to 13 mm.
   Thickness.—Approximately 1 to 2 mm.
   Color.—Pale green (138B and 139C Green Group).
   Leaf glands.—
   Size.—Approximately 1 mm in height and 1 to 1.5 mm in width.
   Number.—Generally 0-2 per leaf.
   Type.—Reniform.
   Color.—Brown (199B of the Grey-Brown Group).
   Leaf stipules.—
   Size.—Medium to large for the species.
   Length.—Approximately 10 to 12 mm.
   Form.—Lanceolate.
   Color.—Green (Yellow-Green 144B-C) with reddish brown tips (Greyed-Orange Group 166C-D and 175A-B) when young. The stipules are considered to be early deciduous.
   Ratio of wood (leaf) buds to flowering buds.—1 to 2 flower buds per vegetative bud.
Flowers:
   Floral buds.—
   General.—The floral buds are considered to be medium to medium large in size, conic in form, and slightly appressed relative to the bearing shoot.
   Color.—The bud scales are silver-brown, (approximately Brown Group 200B-C and Grey Group 201B-C). The buds are considered hardy under typical central San Joaquin Valley, Calif. climatic conditions.
   Length.—Approximately 5 to 7 mm.
   Blooming type.—Considered average in relation to other peach cultivars commonly growing in the central Texas. Date of full bloom was between March 5th and March 17th with an average full bloom date of March 11th during the period between 2006 and 2011 in central Texas.
   Flower type.—Showy.
   Flower size.—Flower diameter at full bloom is approximately 31 to 38 mm.
   Bloom quantity.—Considered abundant.
   Flower bud frequency.—Normally 1 to 2 per node.
   Petal size.—
   General.—Considered medium to medium large for the species.
   Width.—Approximately 11 to 14 mm.
   Length.—Approximately 16 to 21 mm.
   Petal form.—Broadly ovate.
   Petal count.—Nearly always 5.
   Petal color.—Medium pink (Red-Purple Group 62B-C, 65B-D and 73D).
   Petal claw.—
   Form.—The claw is considered truncate in shape and has a medium size when compared to other varieties.
   Length.—Approximately 1 mm.
   Width.—Approximately 1 to mm.
   Petal margins.—Generally considered variable, from nearly smooth to slightly undulate.
   Petal apex.—Generally — The petal apices appear slightly domed.
   Flower pedicel.—
   Length.—Considered medium to long, and having an average length of approximately 5 mm.
   Thickness.—Considered average, approximately 1 to 1.5 mm.
   Color.—A light green (Yellow-Green Group N144A-D).
   Floral nectaries.—
   Color.—Light yellow to white (Yellow Group 8C-D, 10B-D, 11A-B and White Group 155A-B).
   Calyx.—
   Surface texture.—Generally glabrous.
   Color.—A brownish red with green (Red-Purple Group 59B, 60A, Yellow-Green Group N144A-D, Greyed-Purple Group 183A-D, 184 A-B and 185A-B).
   Sepals.—
   Surface texture.—The surface has a short, fine, wooly and a gray-colored texture.
   Size.—Average, and ovate in form.
   Color.—A brownish red with green (Red-Purple Group 59B, 60A, Yellow-Green Group N144A-D, Greyed-Purple Group 183A-D, 184 A-B and 185A-B).
   Anthers.—
   General.—Average in size for the species.
   Color.—Golden yellow (approximately Yellow Orange Group 17A-B).
   Pollen production.—Pollen is abundant, and is a yellow color.
   Filaments.—
   Size.—Variable in length, approximately 12 to 14 mm, with the filaments slightly shorter or equal to the pistil.
   Color.—White with very light pink (Red-Purple 62C-D, 65C-D and White Group 155A-D).
   Pistil.—
   General.—Average in size, but slightly longer or equal to the general anther height.
   Length.—Approximately 15 to 16 mm, including the ovary.
   Color.—Considered a very light green (Yellow-Green Group 145C and 150D).
   Surface texture.—The variety has a long, silver white pubescent pistil (approximately White Group 155A-D).
Fruit:
   Maturity when described.—The present variety of fruit is described, as it would be found in its firm ripe condition at full commercial maturity. Under the ecological conditions prevailing in the medium chill zone of Texas, ‘White Delight Four’ ripens in early July about a month after ‘June Gold’ and 15-20 days after ‘Scarlet Pearl’ in the medium chill zone of Texas.
   Size.—General.—Medium to medium large for the season and considered uniform.
   Average cheek diameter.—Approximately 60 to 66 mm.
   Average suture diameter.—Approximately 64 to 70 mm.
   Average axial diameter.—Approximately 62 to 69 mm.
   Fruit form.—Generally quite round in its lateral aspect. Occasionally, especially under lower chill conditions, the fruit exhibits less symmetry when comparing the suture height with the line opposite the suture. The fruit is generally uniform in symmetry when viewed from the apical aspect.
   Fruit suture.—Generally, the suture appears as a thin line that extends from the base to the apex, and appears deeper at the apex, forming a shallow basin at the apical point. No apparent callusing or stitching exists along the suture line.
   Color.—The suture is normally the same color as the underlying blush, however it has an additional dark red color (Red Group 53B).
   Ventral surface.—Form.—Considered uniform.
   Stem cavity.—
   Size.—Considered moderate for the species.
   Width.—Approximately 14 to 18 mm.
   Length.—Approximately 23 to 28 mm.
   Depth.—Approximately 12 to 17 mm.
   Fruit base.—Round.
   Fruit apex.—Flat.
   Fruit stem.—
   Length.—9-10 mm.
   Thickness.—4-5 mm.
   Color.—Bright green with a yellow green coloration (Green Group 140B and Yellow-Green Group 144B).
   Fruit skin.—Generally considered medium or average in thickness.
   Surface texture.—Thick pubescence.
   Skin acidity.—Considered neutral.
   Tenacious to flesh.—Yes at commercial maturity.
   Tendency to crack.—Not observed.
   Skin color.—Generally — Variable, with approximately 20-40% of the fruit surface covered with an attractive red blush.
   Down.—Thick and long.
   Blush color.—The blush color is generally more prevailing apically. This red blush ranges from a medium red (Red Group 42A) to dark red (Red Group 46A) with many degrees of shading and blending occurring between these colorations.
   Skin ground color.—Very pale yellow with green (Yellow-Green Group 154D).
   Flesh color.—Ivory (White Group 155A).
   Flesh fibers.—Present, numerous and lightly colored. These fibers are present throughout the flesh.
   Stone cavity color.—Medium to light yellow (Yellow Group 1D).
   Flesh texture.—Generally, the flesh is considered firm and fine at commercial maturity.
   Ripening.—Generally the fruit of the present variety ripens evenly.
   Flavor.—Considered very sweet and rich, with a subacid flavor.
   Aroma.—Pleasant and reasonably abundant.
   Eating.—Generally considered very good to excellent.
Stone:
   Attachment.—Clingstone at commercial maturity.
   Stone size.—Generally considered medium-large relative to the ratio of stone to fruit size.
   Length.—Approximately 34 to 36 mm.
   Width.—Approximately 25 to 27 mm.
   Thickness.—Approximately 18 to 21 mm.
   Fibers.—Generally several fibers are attached along the entire surface of the stone.
   Stone form.—Generally the stone is considered elliptical to ovate.
   Stone base angle.—Considered medium.
   Apex shape.—The stone apex is narrow.
   Stone shape.—Considered elongated.
   Stone surface.—
   Surface texture.—Minor surface markings are honeycombed with numerous single pits, rosettes of pits and pit grooves.
   Ventral edge.—Considered medium in size.
   Dorsal edge.—Shape — Grooved and having moderately rough edges.
   Stone color.—The color of the dry stone is medium brown (similar to 165B-C and 173C-D of the Greyed-Orange Group). The color of the inside surface of the endocarp is primarily 164C-D and 165C-D of the Greyed-Orange Group.
   Tendency to split.—no tendency to split.
   Kernel.—the kernel fills the endocarp at harvest and measures approximately 4 to 6 mm in thickness, 25 to 27mm in width, and 15-19 mm in length. When dried the shriveled kernels measure approximately 3-4 mm in thickness, 12-13 mm in width, and 16-19 mm in length. The colors of the shriveled kernels are primarily Greyed-Orange Group 167A-C.
   Use.—The subject variety, ‘White Delight Four’, is considered to be a peach tree of mid-season maturity, which produces subacid white-fleshed fruit which are very firm, attractively colored.
   Keeping quality.—Good.
   Resistance to insects and disease.—No particular susceptibilities were noted.
   Shipping quality.—Average. Although the new variety of peach tree possesses the described characteristics when grown under the ecological conditions prevailing in the medium chill zone of Texas near Fairfield, it will be understood that variations of the usual magnitude and characteristics incident to the changes in growing conditions, fertilization, pruning, and pest control are to be expected.

### REFERENCES

Brooks, R. M. 1958. Double Delight, Earligold, and June Gold peaches. Fruit Var. J. 3:22.

Okie, W. R. 1993. ‘Goldprince’ and ‘Scarletpearl’ peaches. HortScience 28:231.

We claim:

1. A new and distinct Prunus persica tree, substantially as illustrated and described herein.
